(19) United States
(12) Patent Application Publication
(10) Pub. No.: US 2008/0275225 A1
(43) Pub. Date: Nov. 6, 2008

(54) Synthetic Method of 20 (S)-Ginsenoside Rh2

(75) Inventors: Yongzheng Hui, Pudong New Area (CN); Zhiqi Yang, Pudong New Area (CN); Junyao Liu, Pudong New Area (CN); Jijun Teng, Pudong New Area (CN); Huiqin Xie, Pudong New Area (CN); Jie Zhang, Pudong New Area (CN)

Correspondence Address: MORRISON & FOERSTER LLP, 425 MARKET STREET, SAN FRANCISCO, CA, 94105-2482, US

(73) Assignees: SHANGHAI INNIVATIVE RESEARCH CENTER OF TRADITIONAL, Shanghai (CN); SHANGHAI PHARMVALLEY CORP., Shanghai (CN)

(21) Appl. No.: 11/630,751

(22) PCT Filed: May 16, 2005
(86) PCT No.: PCT/CN05/00675

§ 371 Date: Sep. 28, 2007

(30) Foreign Application Priority Data
Jul. 29, 2004 (CN) 200410053269.2

(51) Int. Cl. C07H 15/00

(52) U.S. Cl. 536/6

(57) ABSTRACT

A synthetic method of 20(s)-ginsenoside Rh2, that is 20(S)-protopanaxdiol-3-O-β-D-glucopyranoside, is comprised of: protecting protopanaxdiol (A1) selectively first to produce monosubstituted protopanaxdiol (A2); and Glycosidating the monosubstituted protopanaxdiol with Glucopyranosyl donor in the presence of Lewis acid catalyst; Deprotecting the product; Then separating and purifying to obtain 20(s)-ginsenoside Rh2. The method is conducted under mild condition at low cost, and affords product with high stereoselectivity, high yield and purity. Therefore, the synthetic method of the present invention is suitable for production on large scale.

### FIELD OF THE INVENTION

The present invention relates to a kind of synthetic method of ginsenoside having biologically activity, and particularly relates to a synthetic method of 20(S)-ginsenoside Rh2, named as 20(S)-protopanaxdiol-3-O-β-D-glucopyranoside having the structure of following formula:

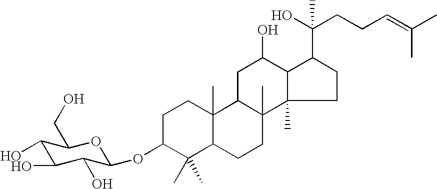

### BACKGROUND OF THE INVENTION

It is well known that the ginseng, whose principal active constituent is ginsenoside, is a valuable Chinese herbal medicine to nourish and build health. 34 kinds of ginsenoside have been discovered until now. Ginsenoside is divided into protopanaxdiol-type ginsenosides, protopanaxtriol-type ginsenosides and oleanolic acid-type ginsenosides according to different ginsengenins. 20(s)-ginsenoside Rh2 belongs to protopanaxdiol-type ginsenosides. In 1985 Odashima from Japan reported (Odashima S et al, Cancer Res. 45(6), 2781, 1985) that 20(s)-ginsenoside Rh2 has effect on inducing the differentiation of melanoma B16 cells, which activates the study in the field all over the world. The follow-up studies proved that 20(s)-ginsenoside Rh2 could be used for suppressing propagation of cancer cell (Ota T et al, Cancer Lett. 110(1-2), 193, 1996) and promoting tumor apoptosis. However, the direct application of ginseng has a limited effect on anti-cancer. One reason is that ginseng contains multiple ginsenoside, among which ginsenoside Rh2 and ginsenoside Rg3 have roles on anti-cancer, while other ginsenoside such as Rg1 and Re have function of promoting synthesis of DNA and RNA and accelerating growth of tumor. Therefore, high pure ginsenoside Rh2 is required in fighting tumor by use of ginseng effectively. The other reason is that white Ginseng contains almost no ginsenoside Rh2 since it is secondary product. After boiling, white Ginseng is changed into Red Ginseng with 0.001% ginsenoside Rh2. But too little content of ginsenoside Rh2 in White Ginseng constrain its direct application in fighting tumor.

The main methods to prepare 20 (S)-ginsenoside Rh2 have been reported as follows:

(1) Enzymolysis (China patent: CN1105781C; Jin Dongshi et al, Journal of Dalian Institute of Light Industry, 2001, 20(2): 99-104.

All kinds of ginseng were hydrolyzed by using ginsenosidase enzyme, such as ginsenoside-glucosidase or -arabinosidase. The part of sugar moiety of ginsenoside was hydrolyzed to obtain Rh2.

Although the biotechnology was used in the process, it took too long time to obtain the ginsenoside glycosidase through culture. Moreover, the product was mixture of many kinds of ginsenosides, with lower yield of 20(S)-ginsenoside Rh2 and higher cost.

(2) Synthesizing 20(S)-ginsenoside Rh2 by use of protopanaxdiol-type ginsenosides as semi-synthetic material:

a. China Patent: CN1091448C, 2002.

20(S)-ginsenoside Rh2 was obtained by the following steps: mixing protopanaxdiol-type ginsenosides solution in water with lower alcohol compound of alkali metal or metal oxide solution in alcohol, or mixing protopanaxdiol-type ginsenosides solution in lower alcohol with alkali metal solution in lower alcohol; allowing reacting at high temperature and high pressure; extracting the product in lower alcohol; purifying the product by silica gel chromatography under low pressure; re-crystallizing the product in methanol/water; obtaining the product of 20(S)-ginsenoside Rh2.

The great disadvantages of the method were that protopanaxdiol-type ginsenosides was needed as initial raw materials, the reaction run under a strict condition of high temperature and high pressure with high cost and low yield of 20(S)-ginsenoside Rh2.

b. Korea Tobacco & Ginseng Central Institute published a preparation method of 20 (R&S)-ginsenoside Rh2 from ginseng. The method comprised the steps as follows: firstly collecting protopanaxdiol-type ginsenosides, hydrolyzing in the presence of acid to collect 20 (R&S)-ginsenoside Rg3, processing 20 (R&S)-ginsenoside Rg3 to obtain ginsenoside Rh2.

The main disadvantages of the method were rare material of protopanaxdiol-type ginsenosides, complicated procedure, large loss of raw material, high cost and low yield rate. Moreover, the product is a mixture of ginsenoside R&S configuration after hydrolyzation.

(3) 20(S)-Ginsenoside Rh2 was Synthesized Using Protopanaxdiol as a Semi-Synthetic Material

a. Japan patent: the Kaiping 8-208688, 1996.

Linear synthetic routes of the method comprised six steps, and were very costly since equivalence of Ag₂CO₃ was used as catalyst in glycosidation. The stereoselection of product does not be conducted effectively. So concerning cost and yield rate, the method was not suitable to be used in large-scale production.

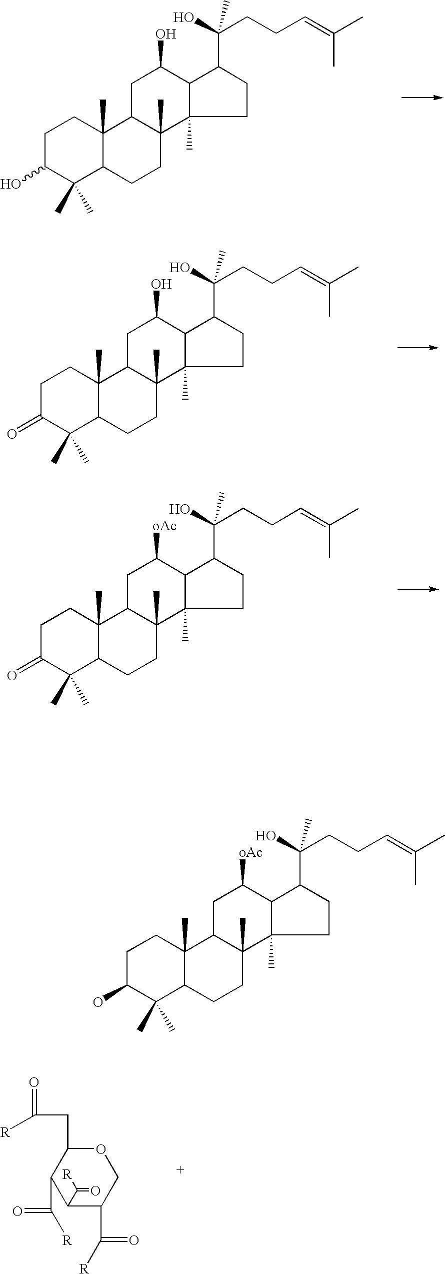

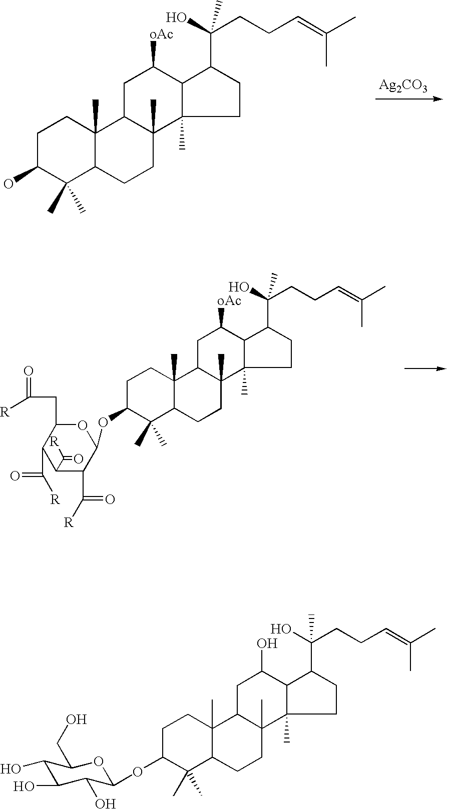

b. Korea Tobacco & Ginseng Central Institute published another method to prepare 20 (S)-ginsenoside Rh2: firstly, 20 (S)-ginsenosidegenin was obtained by hydrolyzing dry powder of leaf and root of ginseng in the presence of strong alkali in alcohol, then 20 (S)-ginsenosidegenin was condensed with glucose in the presence of catalyst, such as AgCO₃, to produce 20 (S)-ginsenoside Rh2.

The method cost much since Ag₂CO₃ was used as a catalyst. Moreover the product was a mixture of two configurations comprising α and β glycosidic bond respectively.

c. Atopkina, L. N., Denisenko, V. A., Novikov, V. L., Uvarova, N. I., CHNCA8, Chem. Nat. Compd. (Engl. Transl.), 1986, 22(3), 279-288

20(S)-ginsenoside Rh2 was obtained by condensation of protopanaxdiol with acetobromo-α-D-glucose in the presence of Ag₂O.

Because hydroxyl groups at 12- and 20-position of protopanaxdiol were not protected, monosubstitution and polysubstitution easily took place by glucopyranosyl during reaction to produce mixed products, comprising five compounds: protopanaxdiol of glucopyranosyl group monosubstituted at 3-, 12- or 20-position respectively, protopanaxdiols of glucopyranoside group disubstituted at 3- and 12-position, and protopanaxdiol of glucopyranoside group disubstituted at 3- and 20-position, among which the protopanaxdiol of glucopyranosyl monosubstituted at 3-position accounts for only 27%. So the target products by this method were difficultly separated with lower yield rate.

The above methods were not suitable for large-scale industrial production because of strict reaction condition, lower yield rate, high cost and ineffective stereoselectivity.

### DETAILED DESCRIPTION OF THE INVENTION

The present invention provides a synthetic method of 20 (S)-ginsenoside Rh2, particular a synthetic method of 20(S)-protopanaxdiol-3-O-β-D-glucopyranoside. The method is described herein with advantages, such as running under mild with low cost, high yield rate, high purity and high selectivity of β-glycosidic bond type product. So the method is suitable for industrial production.

The method of the present invention is illustrated as reaction formula as follows:

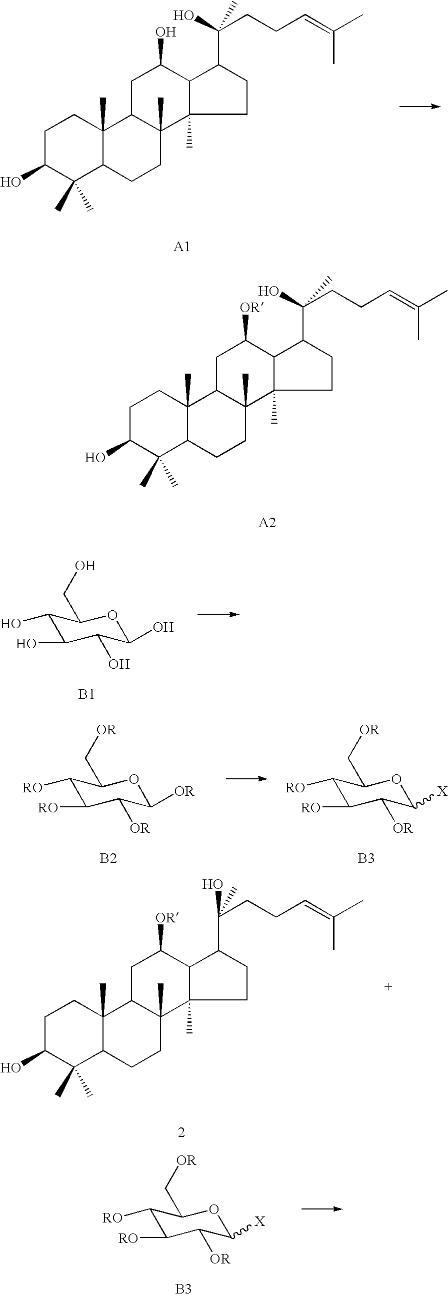

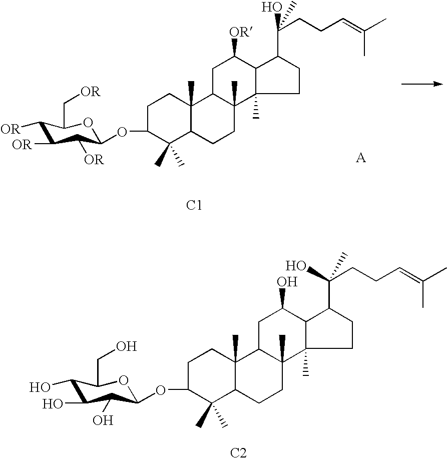

The synthetic method of the present invention is comprised of: protecting protopanaxdiol (A1) selectively to produce monosubstituted protopanaxdiol (A2); Preparing compound (C1) by the reaction of monosubstituted protopanaxdiol (A2) with glucopyranosyl donor compound (B3) in the presence of Lewis acid catalyst and molecular sieve; Purifying compound (C1) by column chromatography or re-crystallization; Obtaining 20 (S)-ginsenoside Rh2 (C2) by the deprotection reaction of compound (C1) and recrystallization.

Particularly, the synthetic method of the present invention is comprised of the following steps:

1. Protecting protopanaxdiol (named herein as A1) selectively to produce monosubstituted protopanaxdiol (named herein as A2), whose structure is

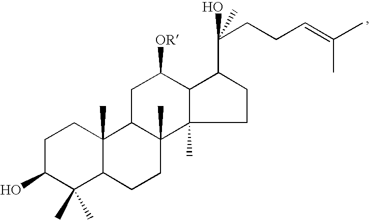

wherein R′ is aromatic hydrocarbons acyl or alkanes substituted aromatic hydrocarbons acyl, C₃-C₆ alkanes substituted acyl, C₃-C₉ alkanes substituted silyl, C₉-C₁₆ aryl substituted silyl, such as benzoyl, p-methoxybenzoyl, pivaloyl, t-butyl-dimethysiyl or t-butyl-diphenylsiyl. The reaction is characterised in that the mole ratio of compound (A1) and reactant with protection groups is 1:3.0-5.0, the reaction runs for 1.5-12 h at −10-25□ in organic solvent with yield percentage of 85-95%, and the organic solvent of reaction is one kind compounds from chloro-alkane, triethylamine, pyridine, N,N-dimethyl formamide, or a mixture of two or more thereof, with amount of 6.5-10 L per mol compound (A1).

2. In the presence of organic solvent and inert gas, compound of polysubstituted 20 (S)-ginsenoside Rh2 (C1), whose structure formula was

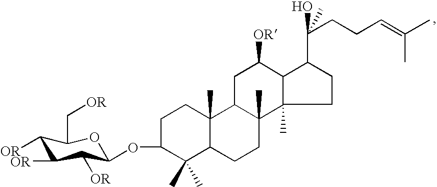

was prepared by the glycosidation reaction of glucopyranosyl donor [compound (B3)], whose structure formula was

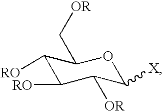

with monosubstituted protopanaxdiol (A2), whose structure formula was

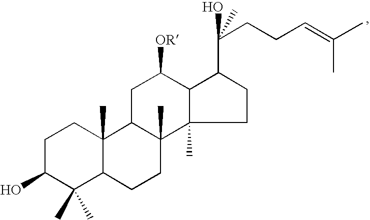

for 0.5-4.5 h under catalyzing by Lewis acid at −20-40□. The molecular sieve may be added to make sure that the reaction was completed thoroughly. After reaction, quencher was added to quench reaction. Compound of polysubstituted 20(S)-ginsenoside Rh2 (C1), whose structure formula is

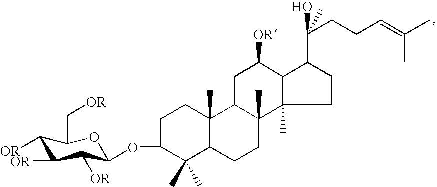

was obtained after solvent was condensed and normally treated.

In the reaction, the mole ratio of compound (A2), compound (B3) and Lewis acid catalyst was 1:0.8-5.0:0.01-1.0. The Lewis catalyst was one compound from chloroacidamide of C₃-C₉, fluoroalkylsulfonyl acid of C₁-C₆, silyl fluoroalkylsulfonate of C₂-C₈, silver fluoroalkylsulfonate of C₁-C₆, boron trifluoride ether complex, for example, N-iodosuccinimide (NIS), mixture of N-iodosuccinimide (NIS)-silver trifluoromethanesulfonate (AgOTf), mixture of N-iodosuccinimide (NIS)-trifluoromethanesulfonate (TfOH), silver trifluoromethanesulfonate (AgOTf), trimethylsilyl trifluoromethanesulfonate (TMSOTf), or a mixture thereof. Molecular sieve was added to promote the speed of the reaction. The said molecular sieve was 3 Å-5 Å the alumina-silicate or their powder. The weight ratio of compound (A2) and the molecular sieve was 1:0-7.0. The solvent was C₂-C₄ chloroalkane or methylphenyl, with amount of 4-12 L per mol compound (A2). The inert gas was nitrogen, argon or helium. Quencher was added to quench reaction. The quencher was trimethylamine, triethylamine or Na₂S₂O₃. The product was purified by column chromatography or re-crystallization. Stuffing of column chromatography was silica gel, aluminum oxide or macroporous resin, et al, And the stuffing preferably was silica gel with granula of 40-60 μm. The weight ratio of silica gel and product was 20-10:1. The elution solvent was one from benzene, dichloromethane, ethyl acetate, chloroform, methanol or cyclohexane. The reacting yield was 70-85%.

In the structure, R′ was aromatic hydrocarbons acyl or alkanes substituted aromatic hydrocarbons acyl, C₃-C₆ alkanes substituted acyl, C₃-C₉ alkanes substituted silyl, C₉-C₁₆ aryl substituted silyl; R was C₂-C₆ alkanes substituted acyl, benzoyl or benzyl; X was OC(NH)CCl₃ or SEt.

3. 20 (S)-ginsenoside Rh2 (C2) was obtained by the deprotection reaction of polysubstituting 20 (S)-ginsenoside Rh2 (C1) with monovalent alkali metal compound in the presence of polar solvent. The monovalent alkali metal compound might be NaOH, sodium methoxide, KOH or LiOH; and concentration of the weight percentage in water was 25-50%; and the mole ratio of compound (C1) and monovalent alkali metal was 1:4-10. The polar solvent was one from tetrahydrofuran, dichloromethane, methanol, ethanol, water or a mixture of two or more thereof, with amount of 10-30 L per mol compound (C1). The reaction was run at temperature of 40-100□ for 10-18 h. Highly purified product of 20(S)-ginsenoside Rh2 (C2) was obtained by re-crystallization in the solvent of one from chloroform, C₁-C₄ alkyl alcohol, ethyl acetate, acetone or water or a mixture of two or more thereof. The yield percentage of the product was 80-90%.

The present invention has advantages of mild condition, simple synthesis routine, cheap raw material, and low cost. In the invention the product of β-glycosidic bond can be obtained with higher selectivity and higher yield rate. Especially, yield rate of the key reaction (glucosides reaction) may reach 70-85%.

Since high purity 20 (S)-ginsenoside Rh2 can be obtained by re-crystallization, the invention method is suitable to be used on a large-scale production.

### EXAMPLES

The present invention will be illustrated by the following non-limiting to further understand it, but they cannot limit content of the present invention.

### Example 1

### Synthesis of Monosubstituted Protopanaxdiol (A2) Having the Structure of the Following

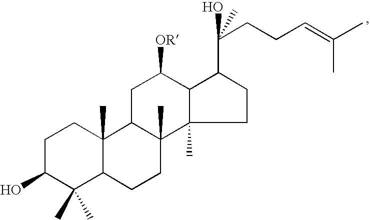

(1) Synthesis of Monosubstituted Protopanaxdiol with R′ of Benzoyl (Bz) (i.e. 12-Benzoyl-Protopanaxdiol)

Protopanaxdiol, which was prepared according to the method described in China patent No. 200410018038.8 (40 g, 0.087 mol), was dissolved in pyridine (600 ml). Benzoyl chloride (44.51 g, 0.261 mol) was added into the protopanaxdiol solution at 0° C. and stirred over night at 25° C. When the reaction was completed, which was determined by thin layer chromatography, methanol was added to terminate reaction. The resulting solid after condensation was dissolved in ethyl acetate, then washed with saturated NaCl solution until to obtain neutral reaction mixture, dried, filtrated, condensed and purified by column chromatography [gradient elution:volume ratio of the benzine and the ethyl acetate was from 6:1 to 3:1] to obtain compound (A2-1) (41.07 g) with yield percentage of 84.3% and purity of 93.63% determined by HPLC.

The physicochemical properties of the compound (A2-1) were as follows:

¹H NMR (300 MHz, CDCl₃): δ7.99-7.37 (m, 5H), 5.2 (m, 1H), 5.13 (t, 1H), 3.9 (dd, 1H), 3.15 (m, 1H), 2.0 (m, 2H), 1.96-1.47 (m, 16H), 1.44-1.24 (m, 8H), 1.16-1.11 (m, 12H)

(2) Synthesis of Monosubstituted Protopanaxdiol with R′ of p-Methoxybenzoyl (MBz) (i.e. 12-p-Methoxybenzoyl-Protopanaxdiol)

Protopanaxdiol, which was prepared according to the method described in China patent No. 200410018038.8 (40 g, 0.087 mol), was dissolved in pyridine (600 ml). MBzCl (59.35 g, 0.348 mol) was added into the protopanaxdiol solution at 0° C. and stirred over night at 20° C. When the reaction was completed, which was determined by thin layer chromatography, methanol was added to terminate reaction. The resulting solid after condensation was dissolved in ethyl acetate, then washed with saturated NaCl solution until to obtain neutral reaction mixture, dried, filtrated, condensed and purified by column chromatography [gradient elution:volume ratio of the benzine and the ethyl acetate was from 8:1 to 3:1] to obtain compound (A2-2) (44.6 g) with yield percentage of 88.6% and purity of 92.26% determined by HPLC.

The physicochemical properties of the compound (A2-2) were as follows:

¹H NMR (300 MHz, CDCl₃): δ8.1-7.86 (m, 4H), 6.85 (m, 4H), 5.13 (t, 1H), 3.84 (t, 6H), 3.2 (s, 1H), 2.15-1.72 (m, 12H), 1.64-1.22 (m, 14H), 1.05 (s, 4H), 1.01 (d, 4H), 0.81 (s, 6H), 0.78 (s, 2H)

(3) Synthesis of Monosubstituted Protopanaxdiol with R′ of Pivaloyl (Piv) (i.e. 2-Pivaloyl-Protopanaxdiol)

Protopanaxdiol, which was prepared according to the method described in China patent No. 200410018038.8 (40 g, 0.087 mol), was dissolved in mixture solution comprising dichloromethane (700 ml) and triethylamine (85 ml). Then pivaloyl (36.5 ml, 0.298 mol) was added into the protopanaxdiol solution. The reaction mixture was cooled down to −10˜5° C. and reacted for 1.5 h. When the reaction was completed, which was determined by thin layer chromatography, methanol was added to terminate reaction. The mixture was washed with saturated NaCl solution, and extracted with dichloromethane. Organic phase was merged, then washed with saturated NaCl solution until to obtain neutral reaction mixture, dried, filtrated, condensed to obtain compound (A2-3) (42.5 g) with yield percentage of 89.7% and purity of 99.48% determined by HPLC.

The physicochemical properties of the compound (A2-3) were as follows:

¹H NMR (300 MHz, CDCl₃): δ5.28 (d, 1H), 3.6 (m, 1H), 3.2 (s, 1H), 2.2-1.8 (m, 6H), 1.72-1.38 (m, 14H), 1.28-1.14 (m, 22H), 1.1 (s, 3H), 0.98-0.72 (m, 9H)

(4) Synthesis of Monosubstituted Protopanaxdiol with R′ of t-Butyl-Dimethylsilyl (TBS) (i.e. 12-t-Butyl-Dimethylsilyl-Protopanaxdiol)

Protopanaxdiol, which was prepared according to the method described in China patent No. 200410018038.8 (40 g, 0.087 mol), was dissolved in mixture solution comprising dichloromethane (700 ml) and triethylamine (70 ml). TBSCl (52.5 g, 0.348 mol) and imidazole (39.7 g, 0.58 mol) was added into the protopanaxdiol solution and stirred for 5 h at 20-250. The reaction was determined to be completed by thin layer chromatography. The mixture was washed with saturated NaCl solution, and extracted with dichloromethane. Organic phase was merged, dried, filtrated, condensed to obtain compound (A2-4) (41.2 g) with yield percentage of 84.5% and purity of 99.21% determined by HPLC.

The physicochemical properties of the compound (A2-4) were as follows:

¹H NMR (300 MHz, CDCl₃): δ5.13 (t, 1H), 3.65 (m, 1H), 3.16 (m, 1H), 2.15-1.72 (m, 6H), 1.67-1.58 (d, 8H), 1.55-1.15 (m, 12H), 1.08 (s, 3H), 0.96 (d, 6H), 0.89 (s, 9H), 0.81 (s, 7H), 0.76 (s, 3H), 0.08 (s, 6H)

(5) Synthesis of Monosubstituted Protopanaxdiol with R′ of t-Butyl-Diphenysilyl (TBDPS) (i.e. 12-t-Butyl-Diphenysilyl-Protopanaxdiol)

Protopanaxdiol, which was prepared according to the method described in China patent No. 200410018038.8 (4 g, 0.0087 mol), was dissolved in N,N-dimethyl-formamide (85 ml). TBDPSCl (11.96 g, 0.0435 mol) and imidazole (3.97 g, 0.058 mol) was added into the protopanaxdiol solution and stirred for 5 h at 20-250. The reaction was determined to be completed by thin layer chromatography. The mixture was washed with saturated NaCl solution, and extracted with dichloromethane. Organic phase was merged, dried, filtrated, condensed and purified by column chromatography [gradient elution:volume ratio of the benzine and the ethyl acetate was from 8:1 to 3:1] to obtain compound (A2-5) (4.9 g) with yield percentage of 82.3% and purity of 99.12% determined by HPLC.

The physicochemical properties of the compound (A2-5) were as follows:

¹H NMR (300 MHz, CDCl₃): δ7.54-7.36 (m, 10H), 5.2 (s, 1H), 3.2 (s, 1H), 3.19 (s, 1H), 1.96-1.71 (m, 8H), 1.56-1.40 (m, 14H), 1.31-1.21 (m, 9H), 1.16-1.11 (m, 9H), 0.86 (t, 9H)

### Example 2

### Synthesis of All-Protected the D-Glucose (B2) Having the Structure of the Following

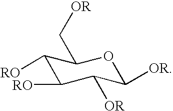

(1) Synthesis of All-Protected the D-Glucose with R of Benzoyl (i.e. 1,2,3,4,6-Five-O-Benzoyl-D-Glucose)

D-glucose (150 g, 0.833 mol) was dissolved in anhydrous pyridine (1650 ml). Benzoyl chloride (532.5 ml, 4.575 mol) was added into the above said solution and stirred at room temperature overnight. When the reaction was completed, which was determined by thin layer chromatography, the reaction mixture was added into a large amount of water. The resulting solid was washed, dried to obtain white solid (B2-1, 552.3 g) with yield percentage of 94.9% and purity of 97.21% determined by HPLC (For synthetic method, see R. K. Ness, et al, J. Amer. Chem. Soc., 1951, 296-299). The physicochemical properties of the compound were as described in Eagle, Andrew J.; et al, J. Chem. Res., 1993, 10, 2663-2679.

(2) Synthesis of All-Protected the D-Glucose with R of Acetyl (i.e. 1,2,3,4,6-Five-O-Acetyl-D-Glucose)

D-glucose (50 g, 28 mmol) and anhydrous sodium acetate (25 g) was added into acetic anhydride (350 ml) and heated to 150-160□ to dissolve the solid. The above solution was injected into ice water and the resulting solid was washed, re-crystallizated in alcohol to obtain compound (B2-2, 86 g) with yield percentage of 79% and purity of 98% determined by HPLC (For synthetic method, see Wolfrom, M. L., Thompson, A. Methods Carbohydr. Chem. 1963, 2, 211). The physicochemical properties of the compound was as described in: Johnson, Carl R. et al, J. Amer. Chem. Soc. 992, 14(24), 9414-9418.

(3) Synthesis of All-Protected the D-Glucose with R of Pivaloyl (i.e. 1,2,3,4,6-Five-O-Pivaloyl-D-Glucose)

D-glucose (1.8 g, 0.01 mol) and 4-Dimethylaminopyridine were dissolved in pyridine (18 ml). Pivaloyl chloride (9 ml, 0.08 mol) was added into the solution drop by drop at 0□ and left for 8 h at 70□. When the reaction was completed, which was determined by thin layer chromatography, white solid of compound (B2-3, 5.53 g) was obtained with yield percentage of 92.13% and purity of 97.62% determined by HPLC (For synthetic method, see BingLi et al, Carbohydrate Research, 2001, 331, 1-7).

The physicochemical properties of the compound were as follows:

¹H NMR (300 MHz, CDCl₃): δ6.66 (d, 1H), 5.25 (t, 2H), 4.78 (dd, 1H), 4.65 (dd, 1H), 4.34 (d, 1H), 4.09 (d, 1H), 1.24 (5×s, 45H, 5C(CH₃)₃CO).

(4) Synthesis of All-Protected the D-Glucose with R of Phenmethyl (i.e. 1,2,3,4,6-Five-O-Phenmethyl-D-Glucose)

D-glucose (1.8 g, 0.01 mol) was dissolved in anhydrous N,N-dimethylformamide (25 ml). NaOH (0.088 g) was added into the solution one by one and stirred continuously for 30 min. Then bromide (0.5 ml) was added drop by drop and stirred overnight at room temperature. When the reaction was completed, which was determined by thin layer chromatography, methanol was added to terminate reaction. The reaction mixture was purified by column chromatography [gradient elution, the benzine and the ethyl acetate volume ratio was from 5:1 to 3:1] to obtain white solid of compound (B24, 5.44 g) with yield percentage of 86.32% and purity of 96.82% determined by HPLC (For synthetic method, see BingLi et al, Carbohydrate Research, 2001, 331, 1-7).

The physicochemical properties of the compound were as follows:

¹H NMR (300 MHz, CDCl₃): δ6.66 (d, 1H), 5.25 (t, 2H), 4.78 (dd, 1H), 4.65 (dd, 1H), 4.34 (d, 1H), 4.09 (d, 1H), 1.24 (5×s, 45H, 5C(CH₃)₃CO).

### Example 3

### Synthesis of Glucopyranosyl Donor (B3) Having the Structure of the Following Formula

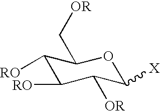

(1) Synthesis of Glucopyranosyl Donor (B3) with R of Benzoyl and X of OC(NH)CCl₃

a. Compound (B2-1) (120 g, 0.146 mol) was dissolved in N,N-dimethylformamide (600 ml). Then glacial acetic acid (20.6 ml, 0.36 mol) was added into the solution when stirring at room temperature. Hydrazine hydrate (20.16 ml, 0.36 mol) was added drop by drop at 0° C. and stirred at room temperature. When the reaction was completed, which was determined by thin layer chromatography, the reaction mixture was purified by column chromatography [gradient elution:the benzine and the ethyl acetate volume ratio was from 5:1 to 3:1] to obtain white solid of compound (66 g) with yield percentage of 64.97% and purity of 95.89% determined by HPLC, having the structure of the following formula:

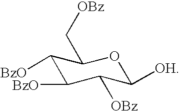

The physicochemical properties of the compound were as described in Mikamo and Masatomo, Carbohydr. Res., 1989, 191: 150-153.

b. The Compound (59 g, 0.095 mol) was obtained in the above process was added into anhydrous dichloromethane (150 ml) and stirred to dissolve the solid. Trichloroacetonitrite (17.37 ml, 0.171 mol) and 1,8-diazabicyclo (5.4.0) undec-7-ene (DBU) (0.708 ml, 4.75 mmol) was added in the presence of argon and stirred for 1.5 h at room temperature. The solution mixture was purified by silica gel column and washed by anhydrous dichloromethane. The solution comprising compound (B3-1) [having the structure of the following formula:

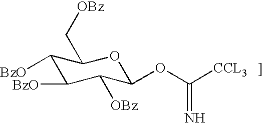

was directly used in following glycosides reaction. (For synthetic method, see Fukase, K., et al, S. Chem. Express. 1993, 8: 409)

(2) Synthesis of Glucopyranosyl Donor (B3) with R of Acetyl and X of OC(NH)CCl₃

a. The compound (B2-2) (3.9 g, 10 mmol) was dissolved in mixture solution THF/MeOH (7:3) (20 ml) which was saturated by ammonia and stirred for 3 h at room temperature. When the reaction was completed, which was determined by thin layer chromatography, the reaction mixture was purified by column chromatography [gradient elution:the benzine and the ethyl acetate volume ratio was from 5:1 to 4:1] to obtain white solid of compound (3.0 g) with yield percentage of 86.17% and purity of 99% determined by HPLC, having the structure of the following formula:

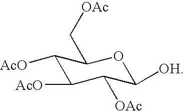

The physicochemical properties of the compound were as described in: Fernandez-Lorente, Tetrahedron, 2003, 59(30): 5705-5712.

b. The compound (3.0 g, 8.62 mmol), which was obtained in the above process, was added into anhydrous dichloromethane (10 ml) and stirred to dissolve the solid. Trichloroacetonitrile (1.6 ml, 15.6 mmol) and K₂CO₃ (0.04 g, 0.4 mmol) was added and stirred for 1.5 h at room temperature in the presence of argon. The solution mixture was purified by silica gel column and washed by anhydrous dichloromethane. The solution comprising compound (B3-2) [having the structure of the following formula:

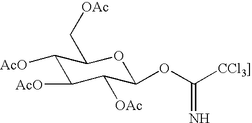

was directly used in the following glycosides reaction.

(3) Synthesis of Glucopyranosyl Donor (B3) with R of Acetyl X of SEt

The compound (B2-2) (7.74 g, 19.8 mmol) was dissolved in anhydrous dichloromethane (47 ml). Then ethanethiol 1.76 ml (23.8 mmol) and anhydrous SnCl₄ 0.35 ml (2.99 mmol) was added. When the reaction was completed, which was determined by thin layer chromatography, the reaction mixture was purified by re-crystallization in ethanol after normal treatment to obtain white solid of compound (B3-3) (6.19 g) with yield percentage of 80%, having the structure of the following formula:

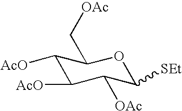

(For synthetic method, see Contour, M. O., et al, Carbohydr. Res., 1989, 193: 283).

The physicochemical properties of the compound (B3-3) were as follows:

¹H NMR (300 MHz, CDCl₃): δ5.22 (t, 1H,), 5.08 (t, 1H), 5.03 (t, 1H), 4.49 (d, 1H), 4.24 (dd, 1H), 4.13 (dd, 1H), 3.71 (ddd, 1H), 2.70 (m, 2H), 2.09, 2.07, 2.04, 2.03 (4×s, 12H, 4CH₃CO), 1.28 (t, 3H).

(4) Synthesis of Glucopyranosyl Donor (B3) with R of Pivaloyl and X of SEt

The compound (B2-3) (5.5 g, 9.16 mmol) was dissolved in anhydrous dichloromethane (35 ml). Ethanethiol (0.81 ml, 10.99 mmol) and anhydrous SnCl₄ (0.16 ml, 1.38 mmol) was added at 0□. When the reaction was completed, which was determined by thin layer chromatography, the white solid (B34) (4.21 g) was obtained followed by normal treatment with yield percentage of 82%, having the structure of the following formula:

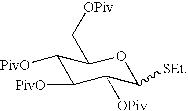

The physicochemical properties of the compound (B34) were as follows:

¹H NMR (300 MHz, CDCl₃): δ5.25-5.23 (m, 2H), 4.93 (t, 1H), 4.78 (t, 1H), 4.65 (t, 1H), 4.34-4.09 (m, 2H), 2.48 (dd, 2H), 1.24 (4×s, 36H, 4C(CH₃)₃CO), 1.2 (m, 3H).

### Example 4

### Synthesis of 20(s)-Ginsenoside Rh2

(1) Synthesis of 20(s)-Ginsenoside Rh2 with R of Benzoyl, R′ of 4-Methoxybenzoyl and X of OC(NH)CCl₃

(a) Glycosidation Reaction

Compound (A2-2, i.e. 12-4-methoxybenzoyl-protopanaxdiol) (3.84 g, 6.24 mmol), compound (B3-1, the liquid was obtained in example 3) (about 24.7 g, 29.95 mmol) and 4 Å molecular sieve 26.3 g were dissolved in anhydrous dichloromethane (75 ml), stirred in presence of nitrogen for 0.5 h. trimethylsilyl trifluoromethanesulfonate (0.06 ml, 0.312 mmol) was added into the above solution and stirred for 0.5 h at 0□. Then triethylamine was added to quench reaction, followed by filtration and condensation. The solution was purified by silica gel column chromatography [eluent:the benzine and the ethyl acetate volume ratio was 6:1] to obtain white solid of compound (C1-1) (4.66 g) with yield percentage of 78.6% and purity of 92.8% determined by HPLC, having the structure of the following formula:

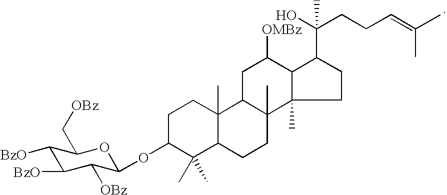

(b) Deprotection Reaction

Compound (C1-1) (4.66 g, 0.004 mol, HPLC: 92.8%) was dissolved in mixed solvent comprising anhydrous dichloromethane (13.35 ml) and ethylalcohol (27 ml). 10 ml sodium methoxide in methanol (comprising 4.32 g, sodium methoxide 50%, 0.04 mol) was added drop by drop under stirring, left for 10 h at 80□. When the reaction was completed, which was determined by thin layer chromatography, the reaction mixture was condensed to obtain white solid. The obtained compound was re-crystallized in mixed solution comprising ethanol and ethyl acetate to obtain compound (C2) (2.13 g) with yield percentage of 85.4% and purity of 99.16% determined by HPLC. The physicochemical properties of the compound was as described in: Chen Yingjie et al, Journal of Shenyang College of Pharmacy, 1987, 11(33): 282-289.

The physicochemical properties of the compound (C2) were as follows:

¹H NMR (300 MHz, C₅D₅N): δ0.89-1.58 (24H, 18-C, 19-C, 21-C, 26-C, 27-C, 28-C, 29-C, 30-C×CH₃), 5.24 (d, 1H), 4.83 (d, 1H), 3.98 (d, 1H), 3.36 (dd, 3H);

¹³C NMR (300 MHz, C₅D₅N): 130.68, 126.3, 106.87, 88.75, 78.7, 78.26, 75.73, 72.91, 71.87, 70.95, 63.07, 56.37, 54.75, 51.68, 50.38, 48.56, 40.0, 39.64, 39.12, 36.95, 35.85, 35.14, 32.03, 31.32, 28.13, 27.07, 26.82, 26.81, 25.76, 23.0, 18.43, 17.64, 17.02, 16.75, 16.32, 15.82;

ESI-MS (m/z): 645.3 (M+Na).

(2) Synthesis of 20(s)-Ginsenoside Rh2 with R of Acetyl, R′ of 4-Methoxybenzoyl and X of SEt

(a) Glycosides Reaction

The compound (A2-2, i.e. 12-p-methoxybenzoyl-protopanaxdiol) (3.84 g, 6.24 mmol), the compound (B3-3) (about 6.19 g, 12.48 mmol) and 5 Å molecular sieve (19.2 g) were dissolved in anhydrous dichloromethane (37.5 ml) and stirred in the presence of argon for 0.5 h. The reaction mixture was cooled down to −20□, and N-iodosuccinimide solid (0.28 g, 1.24 mmol) was added. Then trifluoromethanesulfonate (0.45 ml, 5 mmol) was added drops by drops and stirred for 3 h at 10□. Na₂S₂O₃ was added to quench reaction, followed by filtration and normal treatment. The reaction mixture was purified by re-crystallization in mixed solvent comprising dichloromethane and methanol to obtain white solid of compound (C1-2) (4.22 g) with yield percentage of 71.14% and purity of 94.25% determined by HPLC, having the structure of the following formula:

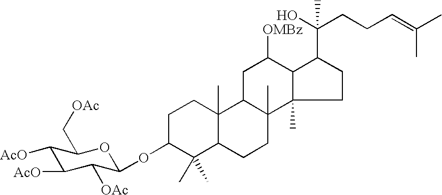

(b) Deprotection Reaction

The compound (C1-2) (3.92 g, 3.83 mmol) was dissolved in mixed solvent comprising tetrahydrofuran (12.8 ml) and ethanol (25.6 ml). 1.3 ml NaOH solution (comprising 0.92 g, 96%, 23 mmol) was added drops and drops under stirring, left for 10 h at 50□. When the reaction was completed, which was determined by thin layer chromatography, the reaction mixture was condensed to obtain white solid. The obtained compound was re-crystallized by acetone to obtain compound (C2) (1.86 g) with yield percentage of 77.1% and purity of 99.43% determined by HPLC.

The physicochemical properties of the compound were described in example 4(1).

(3) Synthesis of 20(s)-Ginsenoside Rh2 with R of Acetyl, R′ of p-Methoxybenzoyl and X of Set

(a) Glycosidation Reaction

Compound (A2-2, i.e. 12-4-methoxybenzoyl-protopanaxdiol) (3.84 g, 6.24 mmol), the compound (B3-3) (about 3.7 g, 7.488 mmol) and 3 Å molecular sieve (8 g) were dissolved in anhydrous dichloromethane (50 ml) and stirred for 0.5 h at room temperature in the presence of nitrogen. The reaction mixture was cooled down to −20□. N-iodosuccinimide solid (0.1 g) was added, and trifluoromethanesulfonate (0.222 ml, 2.5 mmol) was added drops by drops. Na₂S₂O₃ was added to terminate the reaction, followed by filtration and normal treatment. The reaction mixture was purified by Al₂O₃ column chromatography [gradient elution:the volume ratio of benzine and the ethyl acetate was from 8:1 to 5:1] to obtain white solid of compound (C1-3) (4.17 g) with yield percentage of 70.3% and purity of 94.46% determined by HPLC, having the structure of the following formula:

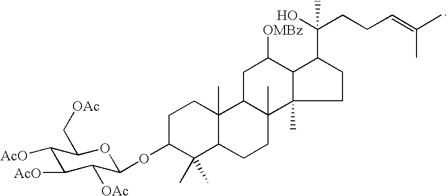

(b) Deprotection Reaction

Compound (C1-3) (0.92 g, 0.9 mmol) was dissolved in mixed solvent comprising tetrahydrofuran (3 ml) and methanol (6 ml). 0.3 ml sodium methoxide (comprising 0.584 g sodium methoxide, 50%, 5.4 mmol) in water was added under stirring at 50%, and left for 18 h. When the reaction was completed, which was determined by thin layer chromatography, the reaction mixture was condensed to obtain white solid. The obtained compound was re-crystallized by mixed solution comprising ethanol and ethyl acetate to obtain compound (C2) (0.46 g) with yield percentage of 81.2% and purity of 99.34% determined by HPLC. The physicochemical properties of the compound were described in example 4(1).

(4) Synthesis of 20(s)-Ginsenoside Rh2 with R of Benzoyl, R′ of Pavaloyl and X of OC(NH)CCl₃

(a) Glycosidation Reaction

Compound (A2-3, i.e. 12-pivaloyl-protopanaxdiol) (42.5 g, 0.0777 mol, HPLC: 99.48%) and compound (B3-1) (about 83.3 g, 0.101 mol, the solution was obtained in example 3) were dissolved in anhydrous dichloromethane, and 4 Å molecular sieve (80 g) was added and stirred for 0.5 h in the presence of argon. trimethylsilyl trifluoromethanesulfonate (1.43 ml, 0.0078 mol) was added drops by drops and stirred for 0.5 h at room temperature. Then trimethylamine (1.2 ml, 0.0086 mol) was added to quench the reaction, followed by filtration and condensation. The mixture solution was purified by silica gel column chromatography [gradient elution:the volume ratio of benzene and the ethyl acetate was 6:1] to obtain white solid of compound (C1-4) (78.5 g) with yield percentage of 82.7% and purity of 91.94% determined by HPLC, having the structure of the following formula:

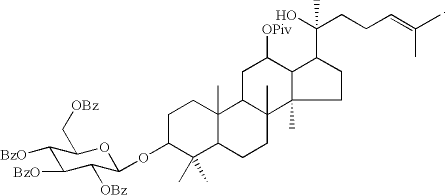

The physicochemical properties of the compound (C1-4) were as follows:

¹H NMR (300 MHz, CDCl₃): δ8.1-7.2 (m, 20H, 4C₆H₅), 5.92 (t, 1H), 4.86 (d, 1H), 5.53 (dd, 2H), 5.14 (s, 1H), 4.82 (d, 2H), 4.48-4.67 (m, 2H), 3.0-3.12 (dd, 1H), 2.32-1.8 (m, 8H), 1.58-1.0 (m, 30H), 0.98-0.72 (m, 9H), 0.65 (d, 6H)

(b) Deprotection Reaction

Compound (C1-4) (78.3 g, 0.064 mol, HPLC: 91.94%) was dissolved in mixed solvent comprising anhydrous dichloromethane (200 ml) and ethanol (700 ml). 45 ml NaOH solution (comprising NaOH 21 g, 96%, 0.504 mol) was added drops by drops under stirring, left for 10 at 40. When the reaction was completed, which was determined by thin layer chromatography, the reaction mixture was condensed to obtain white solid. The obtained compound was re-crystallized by mixed solution comprising ethanol and ethyl acetate to obtain compound (C2) (31.95 g) with yield percentage of 80% and purity of 99.67% determined by HPLC. The physicochemical properties of the compound were described in example 4(1).

(5) Synthesis of 20(s)-Ginsenoside Rh2 with R of R of Acetyl, R′ of Pivaloyl and X of OC(NH)CCl₃

(a) Glycosidation Reaction

Compound (A2-3, i.e. 12-pivaloyl-protopanaxdiol) (3.4 g, 6.24 mmol), compound (B3-2) (about 3.4 g, 6.86 mmol, the solution was obtained in example 3) and 5 Å molecular sieve (8 g) were dissolved in anhydrous dichloromethane. Boron trifluoride (0.08 ml) was added drops by drops in the presence of argon, and stirred for 1.5 h at room temperature. Then trimethylamine was added to quench the reaction, followed by filtration and condensation. The resulting yellow solid (5.9 g) was purified by silica gel column chromatography [gradient elution:the volume ratio of CHCL₃ and CH₃OH is from 10:1 to 7:1] to obtain white solid of compound (C1-4) with yield percentage of 72.6% and purity of 99.8% determined by HPLC, having the structure of the following formula:

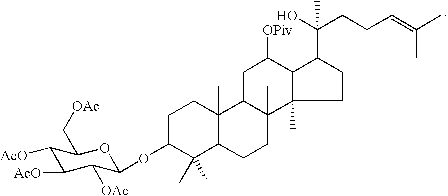

The physicochemical properties of compound (C1-5) were as follows:

¹H NMR (300 MHz, CDCl₃): δ5.21 (t, 1H), 5.14 (m, 3H), 4.81 (dd, 1H), 4.48 (d, 1H), 4.23-4.16 (m, 2H)), 3.08 (m, 1H), 2.67-2.44 (m, 3H), 2.12-2.02 (4×s, 12H, 4CH₃CO), 1.73-1.1.54 (m, 15H), 1.35-1.0 (m, 24H), 0.98-0.72 (m, 9H), 0.65 (d, 6H)

(b) Deprotection Reaction

Compound (C1-5) (0.8 g, 0.9 mmol) was dissolved in mixed solvent comprising dichloromethane (200 ml) and methanol (6 ml). 0.4 ml KOH solution (comprising 0.449 g KOH, 90%, 7.2 mmol) was added drops by drops under stirring, left for 18 h at 50□. When the reaction was completed, which was determined by thin layer chromatography, the reaction mixture was condensed to obtain white solid. The obtained compound was re-crystallized by mixed solution comprising methanol and ethyl acetate to obtain compound (C2) (0.45 g) with yield percentage of 80% and purity of 99.55% determined by HPLC. The physicochemical properties of the compound were described in example 4(1).

(6) Synthesis of 20(s)-Ginsenoside Rh2 with R of Acetyl, R′ of Pivaloyl and X of OC(NH)CCl₃

(a) Glycosidation Reaction

Compound (A2-3, i.e. 12-pivaloyl-protopanaxdiol) (3.4 g, 6.24 mmol) and compound (B3-2) (about 2.47 g, 4.992 mmol, the solution obtained in example 3) were dissolved in anhydrous dichloromethane (25 ml). trimethylsilyl trifluoromethanesulfonate (11.4 ml, 0.0624 mmol) was added drops by drops under stirring in the presence of nitrogen and left for 4.5 h at 35□. Then trimethylamine was added to quench the reaction. The solution was filtrated, absorbed by macroporous resin column, washed by methanol, disabsorpted by cyclohexane. The resulting solution was concentrated to obtain white solid of compound (C1-6) (2.84 g) with yield percentage of 71.2% and purity of 99.2% determined by HPLC, having the structure of the following formula:

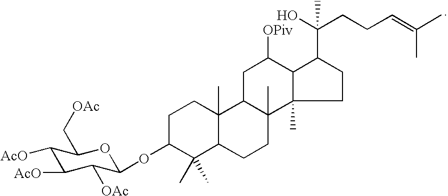

(b) Deprotection Reaction

Compound (C1-6) (2.84 g, 3.19 mmol) was dissolved in mixed solvent comprising tetrahydrofuran (32 ml) and methanol (64 ml). 1.1 ml LiOH solution (containing 0.866 g LiOH, 56%, 12.76 mmol) in water was added drops by drops under stirring, left for 12 h at 50□. When the reaction was completed, which was determined by thin layer chromatography, the reaction mixture was condensed to obtain white solid. The obtained compound was re-crystallized by mixed solution comprising chloroform and acetone to obtain the compound (C2) (1.64 g) with yield percentage of 82.3% and purity of 99.24% determined by HPLC. The physicochemical properties of the compound were described as in example 4(1).

(7) Synthesis of 20(s)-Ginsenoside Rh2 with R of Pivaloyl, R of Pivaloyl and X of SEt

(a) Glycosidation Reaction

The compound (A2-3, i.e. 12-pivaloyl-protopanaxdiol) (5.11 g, 9.375 mmol) and the compound (B3-4) (about 4.2 g, 7.5 mmol) were dissolved in anhydrous dichloromethane and stirred in the presence of helium for 0.5 h at room temperature. The reaction mixture was cooled down to −20□, and N-iodosuccinimide solid (0.15 g) was added. Then 28 ml AgOTf (containing 0.964 g AgOTf, 0.75 mmol) in toluene was added drops by drops and stirred for 2.5 h at 10□. Then Na₂S₂O₃ was added to quench reaction, followed by filtration and normal treatment. The reaction mixture was purified by re-crystallization in mixed solution comprising dichloromethane and methanol to obtain white solid of the compound (C1-7) (5.9 g) with yield percentage of 75.36% and purity of 96.13% determined by HPLC, having the structure of the following formula:

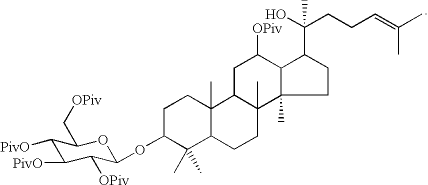

(b) Deprotection Reaction

Compound (C1-6) (5.9 g, 5.66 mmol) was dissolved in mixed solvent comprising tetrahydrofuran (19.2 ml) and ethanol (38.4 ml). 2 ml KOH solution (containing 2.12 g KOH, 90%, 33.96 mmol) was added drops by drops under stirring, and left for 12 h at 55□. When the reaction was completed, which was determined by thin layer chromatography, the reaction mixture was condensed to obtain white solid. The obtained compound was re-crystallized in mixed solution comprising methanol and ethyl acetate to obtain the compound (C2) (2.9 g) with yield percentage of 82.3% and purity of 99.24% determined by HPLC. The physicochemical properties of the compound were described as in example 4(1).

1. A synthetic method of 20(s)-ginsenoside Rh2, which use protopanaxdiol as a raw material, characterised in comprising steps as follows:
(a) Protecting Protopanaxdiol (named herein as A1) selectively to produce monosubstituted protopanaxdiol (named herein as A2), whose structure is 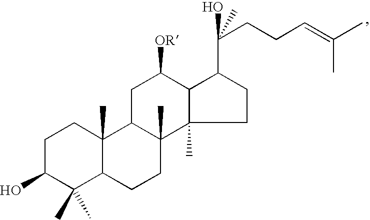
wherein R′ is aromatic hydrocarbons acyl or alkanes substituted aromatic hydrocarbons acyl, C₃-C₆ alkanes substituted acyl, C₃-C₉ alkane substituted silyl, C₉-C₁₆ aryl substituted silyl, such as benzoyl, methoxybenzoyl, pivaloyl, t-butyl-dimethysiyl or t-butyl-diphenylsiyl, characterised of the reaction in that the mole ratio of compound (A1) and reactant with protection groups is 1:3.0-5.0, the reaction runs for 1.5-12 h at −10-250 in organic solvent, and the organic solvent of reaction is one kind compounds from chloro-alkane, triethylamine, pyridine, N,N-dimethyl formamide, or a mixture of two or more thereof, with amount of 6.5-10 L per mol compound (A1). The yield of the reaction was 85-95%.
(b) Preparing polysubstituted 20 (S)-ginsenoside Rh2 [compound (C1)], whose structure formula is 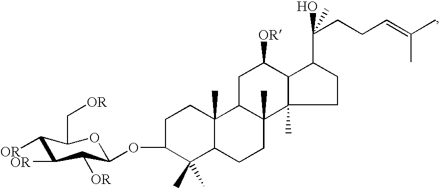
by the glycosidation reaction of monosubstituted protopanaxdiol (A2), whose structure formula is 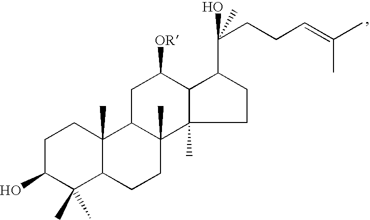
with glucopyranosyl donor [compound (B3)], whose structure formula is 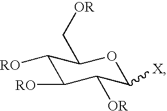
Lewis acid catalyst and molecular sieve in organic solvent in the presence of inert gas, wherein R′ is aromatic hydrocarbons acyl or alkanes substituted aromatic hydrocarbons acyl, C₃-C₆ alkanes substituted acyl, C₃-C₉ alkanes substituted silyl, C₉-C₁₆ aryl substituted silyl; R is C₂-C₆ alkanes substituted acyl, benzoyl or benzyl; X is OC(NH)CCl₃ or SEt, characterised of the glycosidation reaction in that the mole ratio of compound (A2)/compound (B3)/Lewis acid catalyst is 1:0.8-5.0:0.01-1.0, the weight ratio of compound (A2)/molecular sieve is 1:0-7.0, the reaction temperature is −20-40□, the reaction time is 0.5-4.5 h, the amount of reaction solvent is about 4-12 L per mol compound (A2), quencher is added to quench reaction after reaction, the product is purified by column chromatography analysis or re-crystallization.
(c) Obtaining 20 (S)-ginsenoside Rh2 (C2) by the deprotection reaction of compound (C1) with compound monovalent alkali metal compound in the presence of polar solvent, characterised of the reaction in that the mole ratio of compound (C1) and compound monovalent alkali is metal compound 1: 4-10, the reaction temperature is 40-100□, the reaction time is 10-18 h, the amount of polar solvent is 10-30 L per mol compound (C1), and the product is purified by re-crystallization.

2. The synthetic method according to claim 1, characterized in that the said Lewis acid catalyst is one compound from chloroacidamide of C₃-C₉, fluoroalkylsulfonyl acid of C₁-C₆, silyl fluoroalkylsulfonate of C₂-C₈, silver fluoroalkylsulfonate of C₁-C₆, boron trifluoride ether complex or theirs mixture in the glycosidation reaction.

3. The synthetic method according to claim 1, characterized in that the inert gas is nitrogen, argon or helium in glycosidation reaction.

4. The synthetic method according to claim 1, characterized in that organic solvent is C₂-C₄ chloroalkane or methylphenyl in glycosidation reaction.

5. The synthetic method according to claim 1, characterized in that quencher is trimethylamine, triethylamine or Na₂S₂O₃ in glycosidation reaction.

6. The synthetic method according to claim 1, characterized in that molecular sieve is 3 Å-5 Å alumina-silicate or theirs powder in glycosidation reaction.

7. The synthetic method according to claim 1, characterized in that stuffing of column chromatography was silica gel, aluminum oxide or macroporous resin in glycosidation reaction.

8. The synthetic method according to claim 1, characterized in that the eluent used the column chromatography is one compound from benzine, dichloromethane, ethyl acetate, chloroform methanol and cyclohexane, or a mixture thereof in glycosidation reaction.

9. The synthetic method according to claim 1, characterized in that the compound monovalent alkali metal compound is NaOH, sodium methoxide, KOH or LiOH in deprotection reaction.

10. The synthetic method according to claim 1, characterized in that the polar solvent is one from chloroform, methanol, dichloromethane, ethanol and water, or a mixture of two or more thereof in
